(19) United States
(12) Patent Application Publication
(10) Pub. No.: US 2015/0203594 A1
(43) Pub. Date: Jul. 23, 2015

(54) Topographically Functionalized NFC Film as an Immunoassay Platform for Rapid Diagnostics

(71) Applicant: Aalto University Foundation, Aalto (FI)

(72) Inventors: Hannes Orelma, Aalto (FI); Ilari Filpponen, Aalto (FI); Janne Laine, Aalto (FI)

(73) Assignee: Aalto University Foundation, Aalto (FI)

(21) Appl. No.: 14/410,409

(22) PCT Filed: Jun. 28, 2013
(86) PCT No.: PCT/FI2013/050721

§ 371 Date: Dec. 22, 2014

Related U.S. Application Data
(60) Provisional application No. 61/665,328, filed on Jun. 28, 2012.

(51) Int. Cl. C08B 3/00; C07K 16/18; C07K 14/765

(52) U.S. Cl. CPC C08B 3/00; C07K 14/765; C07K 16/18

(57) ABSTRACT

The present invention concerns a method for functionalization, via topographical modification, of the surfaces of nanofibrillated (NFC) cellulose films into non-porous, water-resistant platforms, usable in diagnostic applications. The method includes a carboxylation of the NFC-film via TEMPO-mediated oxidation, and optionally an activation via EDS/NHS chemistry and, finally, the reactivity of the film can be tested using anti-human IgG. The invention also concerns the thus prepared functionalized NFC films, as well as the use thereof as platforms for diagnostical assays.

### BACKGROUND OF THE INVENTION

1. Field of the Invention

The present invention concerns a method for topographical modification of nanofibrillar cellulose (NFC) films that have been activated with TEMPO-oxidation, as well as the use of these films as a platform for, e.g. diagnostical assays.

2. Description of Related Art

Polymeric materials (such as plastics) made of fossil sources have been utilized in different medical and diagnostical applications as a nonporous support material (see e.g. Wu P., et al., 2008; Yager P., et al., 2006; Lequin R. M., 2005). However, these plastics are non-renewable oil based materials, the commercial utilization of which in large scale must be considered harmful for the environment.

Cellulose and cellulose derivatives could be a viable alternative, due to their inherent properties such as non-toxicity, hydrophilicity, and chemical resistivity (see e.g. Siró I. and Plackett D., 2010; Pelton R., 2009), but they tend to have a high porosity that makes them unpractical. Of the cellulose derivatives, for example nitrocellulose has been used as a supporting material (see e.g. Mansfield M. A., 2005), here for conjugating antibodies in diagnostic assays. However, nitrocellulose has certain disadvantages such as non-optimal mechanical strength, flammability, and the affinity to adsorb proteins non-specifically, due to its cationic charge.

Nanofibrillated cellulose (NFC), produced from wood fibres via mechanical or chemical disintegration (such as in Saito T., et al., 2007; and Pääkkö M., et al., 2007), exhibits interesting properties such as high surface area, high hydrogen bonding ability, and good mechanical strength (see e.g. Syverud K. and Stenius P., 2009; and Yano H. and Nakahara, S., 2004). This material can be used to manufacture strong and chemically resistant materials.

Recently, NFC-gel has been used to manufacture nearly transparent and smooth films (see e.g. Karabulut E. and Wagberg L., 2011; Spence K., et al., 2010; and Nogi M., et al., 2009). These films can be improved by using low charged pulp as raw material. This significantly reduces the water impregnation, probably because of the small porosity and compact structure of NFC-films (Osterberg M., et al., 2013). Water resistant NFC-films would be useful in the diagnostical applications where the penetration of analyte in the supporting material is undesired. Moreover, the hydrophilicity of such NFC-films could be beneficial for reducing proteins non-specific adsorption (see e.g. Norde W., 1996).

Derivatisation of NFC via TEMPO-mediated oxidation is a technique that is used to selectively convert the primary hydroxyls of cellulose to their carboxyl form, which could be further utilized in various applications, such as immunoassays (see e.g. Saito T., et al., 2007; De Nooy A., et al., 1995; Isogai A. and Kato Y., 1998; Saito T., et al., 2006; Saito T. and Isogai A., 2004; Habibi Y., et al., 2006).

The oxidation reaction occurs more rapidly at the regions that are easily accessible for the oxidation chemicals. TEMPO-oxidized NFC-gel has been used in highly transparent films with low air permeability (see e.g. Fukuzumi H., et al., 2009), but unfortunately these films have quite low water resistance, due to the high homogenously distributed surface charge of the oxidized cellulose nanofibrils.

Surface specific oxidation of water resistant NFC-films could offer an alternative pathway to produce immunodiagnostic platforms with low porosity, high stability and suitable conjugation sites for immobilization of proteins. The compact and closed structure of these NFC-films limits the amount of diffused water which in turn diminishes the oxidation reaction inside the film's structure (see e.g. Spence K., et al., 2010).

Utilization of the inkjet printing technique on the deposition of biosensors and immunoassays on paper and other supports has been widely reported (see e.g. Abe K., et al., 2008; Hossain S. M., et al., 2009; Di Risio S. and Yan N. J., 2010; Lonini L., et al., 2008). The main advantages of the inkjet printing technique are the small demand of printing liquids (expensive purified antibodies and enzymes can be used; as in Delaney J. T., et al., 2009), and the lack of physical contact with the object, which reduces the risk of the surface contamination. Moreover, the small droplet size (in the scale of pico liters), allows for controlling the surface concentration of sensing molecules (see e.g. Rose D., 1999).

Traditionally, functionalized cellulose films are, however, still manufactured from prefunctionalized raw materials. This functionalization takes place also in the inner parts of cellulose film that promotes its stability in water, whereas a topographic surface modification would allow a functionalization of only the surface.

### SUMMARY OF THE INVENTION

It is an object of the present invention to provide a procedure for topographically modifying only the surfaces of nanocellulose films.

Particularly, it is an object of the present invention to provide surface-modified nanocellulose films suitable for use in producing various functional surfaces, e.g. barrier films, bioactive indicators, and affinity filters.

These and other objects, together with the advantages thereof, over known methods, functionalized films and uses thereof, are achieved by the present invention, as hereinafter described and claimed.

Thus, a method for functionalization of nanofibrillated (NFC) cellulose films into non-porous, water-resistant platforms, usable in diagnostic applications, is presented.

The topographic surface modification of the present invention allows functionalizing only the surfaces of the nanocellulose films, leaving the inner area to native form. The topographical carboxylation allows increasing the hydrophilicity of surfaces of NFC-films, and allowing conjugating specific molecules on these surfaces.

The present invention concerns a method for topographical modification of water resistant nanofibrillar cellulose (NFC) films with TEMPO-oxidation, particularly to provide a platform of diagnostical assays, or to provide a biofilter.

More specifically, the method of the present invention is characterized by what is stated in the characterizing part of claim 1.

Further, the functionalized film of the present invention is characterized by what is stated in claim 10 and the use of such films and methods is characterized by what is stated in claim 11.

Considerable advantages are achieved using the present invention. For example, the results described below have shown the potential of the activated NFC-films utilized as a supporting material for diagnostical applications.

The used surface modification techniques are sensitive, and maintain the high strength and resistance properties of NFC films, as well as the low toxicity.

Further, the topographical carboxylation produces a functional film structure where the native cellulose in the film enhances the water resistance, whereas the increased hydrophilicity on the surfaces of the film leads to lower non-specifically binding of detected biomolecules.

The thus developed biointerface can be utilized to detect positively charged molecules, which in many cases is challenging.

### BRIEF DESCRIPTION OF THE DRAWINGS

FIG. 1 is a schematic illustration for topographical modification of nanofibrillar cellulose film with TEMPO-mediated oxidation followed by EDC/NHS activation. Antibodies can be installed on the activated NFC-film by either inkjet printing or physical adsorption.

FIG. 2 shows the confocal laser scanning microscopy (CLSM) image for an unmodified NFC-film (see FIG. 2a). The reflection mode with topographical imaging mode was used. A digital photograph of an unmodified NFC-film size of 4.5×2.5 cm² is also shown (see FIG. 2b).

FIG. 3 shows the effect of the TEMPO-mediated oxidation on contact angle and charge of NFC-films as a function of the TEMPO-oxidation time. Contact angles are obtained after 10 sec recording.

FIG. 4 shows the QCM-D adsorption curve for sequential TEMPO-oxidation and EDC/NHS activation of a NFC-surface (see FIG. 4a), as well as the adsorption of 100 μg/ml BSA in 10 mM NaOAc at pH 5 on unmodified NFC, TEMPO-oxidized NFC, and TEMPO-oxidized NFC with EDC/NHS activation (see FIG. 4b). EDC/NHS activated NFC-film was allowed to dry in a desiccator over night before the adsorption experiment. All adsorption curves are obtained using fifth normalized overtones.

FIG. 5 shows the XPS spectra for unmodified, TEMPO-oxidized, TEMPO plus EDC/NHS activated NFC-films, and TEMPO plus EDC/NHS activated NFC-film after immobilization of human IgG.

FIG. 6 shows the AFM images of an unmodified NFC-film (see FIG. 6a), a TEMPO-oxidized NFC-film (see FIG. 6b), and an NHS-activated NFC-film (see FIG. 6c). TEMPO-oxidation time of 120 seconds was used to obtain FIGS. 6b and 6c.

FIG. 7 shows AFM images of the unmodified NFC-film (FIG. 7a), TEMPO-oxidated NFC-film (FIG. 7b), the EDC/NHS activated NFC-film (FIG. 7c), and printed anti-hIgG on the EDC/NHS activated NFC-film (FIG. 7d), with adsorbed antihuman IgG on the EDC/NHS activated NFC-film. TEMPO-oxidation time was 120 sec.

FIG. 8 shows phase images for the unmodified NFC-film (FIG. 8a), TEMPO-oxidized NFC-film (FIG. 8b) and EDC/NHS activated NFC-film (FIG. 8c).

FIG. 9 shows CLSM intensity images for adsorbed FITC-stained anti-human IgG (0.1 mg/ml in 10 mM phosphate buffer at pH 7.4) on an unmodified NFC-film (see FIG. 9a) and an activated NFC-film (film treated with 120 sec. TEMPO-oxidation plus EDC/NHS activation) (see FIG. 9b) with 10 mM NaCl at pH 10 rinsing for removing electrostatically bound antibodies. Both images are recorded using 848 V laser power with constant imaging conditions.

FIG. 10 shows the QCM-D data for measurement of activity of conjugated anti-human IgG (0.1 mg/ml in 10 mM NaOAc at pH 5) on the activated NFC surface.

FIG. 11 shows QCM-D data of the adsorption of human IgG (hIgG) on EDC/NHS activated NFC-film without antihuman IgG.

FIG. 12 shows an image of printed dansylated anti-human IgG (1 mg/ml in 10 mM phosphate buffer at pH 7.4) on an activated NFC-film (120 sec. TEMPO-oxidation plus EDC/NHS activation) under UV-light (366 nm) (see FIG. 12a), and a CLSM intensity image for printed FITC-stained anti-human IgG (1 mg/ml in 10 mM phosphate buffer at pH 7.4) on an activated NFC-film by CLSM (see FIG. 12b). The images were recorded using 848 V laser power. Also an AFM height image on printed anti-human IgG on an activated NFC-film is shown (see FIG. 12c). The z-scale of FIG. 12c is 50 nm.

### DETAILED DESCRIPTION OF PREFERRED EMBODIMENTS OF THE INVENTION

The present invention concerns a method for topographical modification of water resistant nanofibrillar cellulose (NFC) films with TEMPO-oxidation. This reaction step, as such, already produces some topographical changes in the surface.

Thus, in the present invention, NFC-films are first TEMPO-oxidized to introduce new functional (carboxylic) groups on the film's surface.

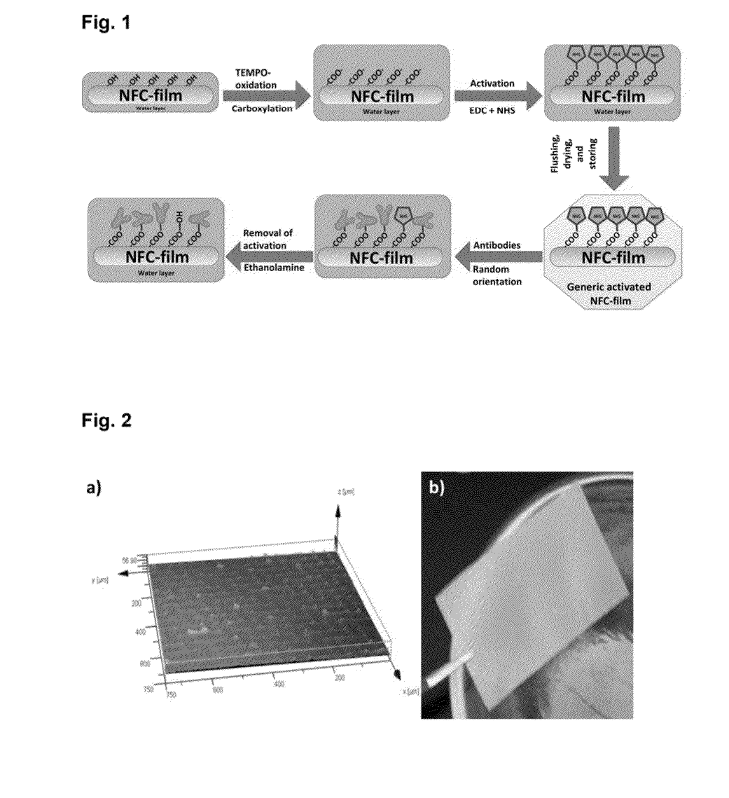

An exemplary embodiment of the invention has been illustrated in FIG. 1.

The raw materials used to prepare the topographically modified films of the present invention are completely renewable. Further, the use of organic solvents can be avoided, since the entire method can be carried out in aqueous media.

Preferably, the NFC film used as the substrate is prepared from mechanically disintegrated cellulose gel. This gives a water resistant NFC-film.

The mentioned TEMPO oxidation is generally carried out by adding the NFC film into an aqueous solution (including the carboxylation chemicals), which has a slightly alkaline pH, such as a pH of 8-12, preferably about 10. The reaction kinetics of the reaction is extremely fast that allows use short reaction time that prevents the diffusion of chemicals in the NFC-films. Also the structure of NFC-films decreases the accessibility of reaction chemicals to diffuse in the film.

Due to the fast reaction kinetics, a maximum amount of carboxyl groups should have been generated in less than 10 minutes, whereby the reaction can be stopped already after a time of, for example 10 to 300 sec. The reaction is stopped, for example by the addition of ethanol and by washing to remove the carboxylation chemicals.

According to a preferred embodiment of the invention, one or more further surface modification steps are carried out subsequent to the carboxylation.

Said further surface modification steps can include, for example a functionalization step, wherein the carboxyl groups are further reacted and converted into amine-reactive ester groups using an EDC/NHS reaction. This reaction will change the NFC surface into a more amine-reactive form.

According to this alternative, carboxylic groups are used as reactive sites for further activation via EDC/NHS chemistry that leads to amine reactive NFC surfaces which are then subsequently activated by EDC/NHS chemistry for introducing amino reactive NHS-esters on the NFC-film (see FIG. 1).

This esterification is generally carried out in an aqueous solution (including the EDC and the NHS) at a slightly acidic pH, such as a pH of 4-6, preferably about 5. The reaction is relatively fast, and a maximum amount of ester groups should have been generated in less than 30 minutes.

Subsequently, the thus activated NFC-films can be used for supporting materials for conjugated molecules. Thereby, an optional conjugation of DNA, antibodies or proteins on the activated NFC-films can be carried out, as demonstrated in the examples below using polyclonal anti-human IgG and bovine serum albumin (BSA) by inkjet and adsorption methods.

Thus, either alternatively or additionally (particularly subsequent to the optional esterification step), the further surface modification steps can include, for example a step wherein the prepared surface-reactive NFC film is modified by attaching amine-containing molecules, such as biomolecules (including proteins, DNA and antibodies) to the carboxyl groups formed on the surface of the film during the oxidative activation.

The amine-containing molecules can be selected from, e.g. biomolecules, preferably from proteins, DNA and antibodies, more preferably from either water-soluble proteins, such as albumins, or from monomeric immunoglobulins, such as IgG, IgD and IgE, most suitably from bovine serum albumin (BSA) or an immunoglobulin IgG. Generally, these amine-containing molecules will attach to either the carboxyl groups or the ester groups on the NFC surface.

The application of the amine-containing molecules onto the surface of the NFC film is preferably realized using non-contact application methods, more preferably selected from ink-jet printing, spraying and adsorption techniques.

Particularly inkjet printing has been found to be a suitable technique to be utilized in such a topographic modification to deposit sensing molecules to the interface of the surface of a suitable support, such as a nanocellulose film.

The present invention also concerns a nanofibrillar cellulose (NFC) film with a topographically modified surface, prepared as described above.

The activated topographical films that are formed using the present invention are suitable for use in producing various functional surfaces, e.g. barrier films, bioactive indicators, and affinity filters, particularly for use as platforms for diagnostical assays, and as biofilters.

This developed invention allows to functionalize also other cellulosic materials such as paper, films, membranes, etc.

The following non-limiting examples are intended merely to illustrate the advantages obtained with the embodiments of the present invention.

### EXAMPLES

The materials used in the following experiments were: anti-human IgG (#19764), human IgG (#14506), NHS (N-hydroxysuccinimide, #130672), EDC (1-ethyl-3-[3-dimethylaminopropyl]carbodiimide hydrochloride, #03450), ethanolamine (#398136), BSA (Bovine Serum Albumin, #A7030), dansyl chloride (#39220), FITC (Fluorescein 5(6)-isothiocyanate, #46950), and TEMPO free radical ((2,2,6,6-Tetramethyl-piperidin-1-yl)oxyl, #426369) were obtained from Sigma-Aldrich (Helsinki, Finland).

The water used in all experiments was deionized and further purified with a Millipore Synergy UV unit. All other chemicals were laboratory scale, and used without any purification steps. An Epson R800 piezoelectric inkjet printer with a CD-printing tray was used without any modifications. Clean unused inkjet cartridges were obtained from MIS Associates, MI, USA.

The preparation mechanisms and chemical and topographical changes on the NFC-films were demonstrated by using QCM-D, contact angle, XPS, CLSM, AFM and conductometric titration techniques.

Particularly, the surface carboxylation, activation and the protein adsorption were confirmed using contact angle measurements, conductometric titrations, X-ray photoelectron spectroscopy (XPS) and fluorescence microscopy. Surface morphology of the films was analysed with confocal microscopy and atomic force microscopy (AFM). All reactions were monitored in-situ using a Quartz Crystal Microbalance with Dissipation Monitoring (QCM-D) on nanocellulose model surfaces prepared from the same NFC gel. The amine reactivity of the prepared biointerface after drying and storage was demonstrated with BSA. Furthermore, the anti-human IgG could be installed on the activated NFC-film with commercial piezoelectric inkjet printing. It was thus demonstrated that the developed biointerface can be used as a generic, environmentally sustainable immunoassay platform for rapid diagnostic applications.

### Example 1

### Preparation of Nanofibrillar Cellulose (NFC) Films and Model Cellulose Surfaces

The NFC-films used in this work were prepared as presented by Osterberg et al. (Osterberg et al, submitted). Briefly: five times masuko grinded bleached hardwood pulp (Birch) was disintegrated by a M110P plug and play fluidizer (Microfluidics corp., Newton, Mass., USA) with six passes. The NFC-gel was then fibraged on the filter membrane using 2.5 bar pressure to remove the excess water. The deposited film was then rolled five times with a smooth metal rolling pin to tight the structure of the film. Finally prepared films were dried between clean plotting boards under external pressing. The prepared NFC-films were used without any pretreatment steps, and stored at the room atmosphere.

The NFC-model cellulose surfaces were prepared as is described by Ahola et al. (2008). Briefly: five times masuko grinded NFC-gel from the bleached suphite hardwood pulp (Birch) was disintegrated by using a M110P plug and play fluidizer (Microfluics corp., Newton, U.S.) with 20 passes. The individual cellulose nanofibrils were then produced by a mechanical stirring with an ultrasonic microtip for 10 min with 25% amplitude settings, and the centrifugation at 10400 rpm for 45 min, respectively. Individualized cellulose nanofibrils were then collected from the supernatant by manual pipetting. The individualized NFC (0.148 w-% NFC in MilliQ-water) was then spin coated on PEI-modified SiO₂-crystals with a WS-650SX-6NPP spin coater (Laurell Technologies, North Wales, Pa., USA) using a spin-coating condition of 3000 rpm with a 1.5 min spinning time. The NFC-coated QCM-D crystals were stored at a desiccator. Prior the QCM-D measurements the NFC-surfaces were allowed to stabilize overnight in MilliQ-water.

### Example 2

### Topographical Activation of the NFC Films by TEMPO-Mediated Oxidation and EDC/NHS Treatment

The unmodified NFC-films were functionalized by using the 2,2,6,6,-tetramethylpipelidine-1-oxyl radical (TEMPO)—NaBr—NaClO system as described Isogai et al. (1998). Briefly: 0.13 mmol TEMPO and 4.7 mmol NaBr were dissolved in 100 ml of MilliQ-purified water. Then 5.65 mmol NaClO was added in the solution, and the pH of the solution was adjusted to 10 by adding 1M HCl. The NFC-films, size 2.5×2.5 cm², were placed in a glass petri dish, and the TEMPO solution was added on the NFC-films. The NFC-films were kept in the solution varying times from 10 to 300 sec, and the oxidation reaction was stopped by an ethanol addition and washing with the large extent of MilliQ purified water, respectively, to remove carboxylation chemicals. The carboxylated NFC-films were transferred to amine reactive via an EDC/NHS activation. Carboxylated NFC-films were kept in a solution of 0.1 M EDC plus 0.4 M NHS in 10 mM NAOAc buffer pH 5 for 20 min, and rinsed with Milli-Q water. The topographically activated NFC-films were then dried between clean plotting boards for preventing the buckling of the films. The procedure has been briefly illustrated in new FIG. 1.

The water tolerance of the prepared unmodified NFC-films was verified in tests in which the films were immersed in Milli-Q water for several days. No visible changes on the films were observed afterwards (FIG. 2b), suggesting that the pressure filtered low-charged NFC films have satisfactory strength properties also in the wet state (Österberg et al, submitted).

### Example 3

### Analysis of the Unmodified and the Activated Films

The macro scale topography of the unmodified NFC-film was characterized by CLSM (FIG. 2a). The wire markings from the filtration membrane are clearly visible, while no macroscale voids or pores were observed, suggesting a uniform NFC-film surface, with RMS roughness of about 6.8±1.0 μm. The low porosity and the water resistance are expected to reduce the diffusion of biomolecules into the NFC-film, enhancing the specificity of the immunoassay.

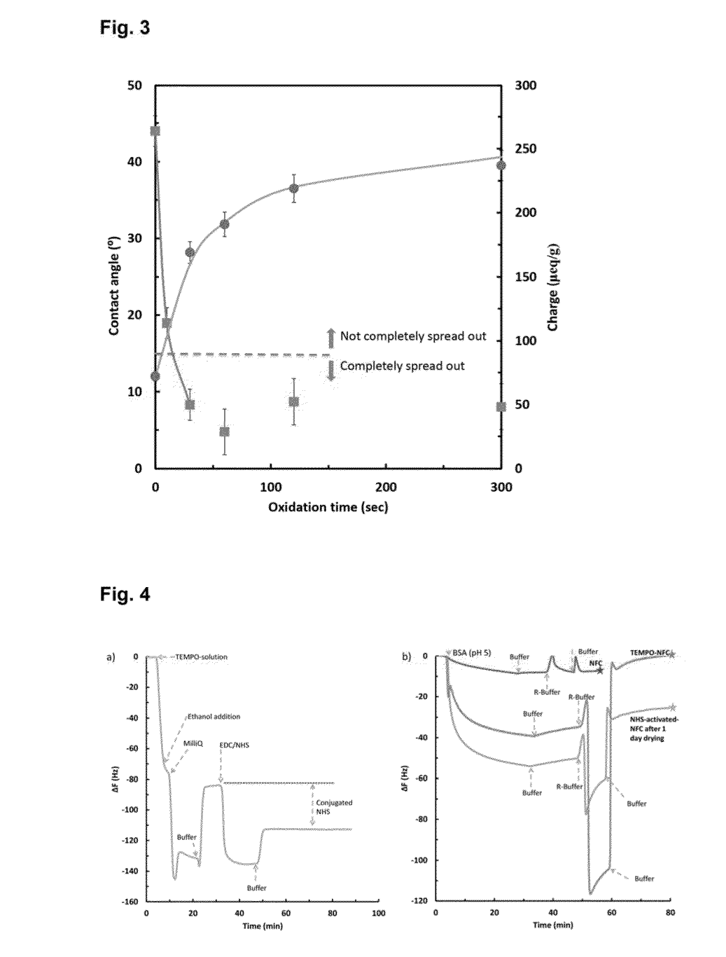

The substantial increase of carboxyl groups on the surface of TEMPO-oxidized NFC-films was verified with by conductometric and contact angle measurements (FIG. 3). This analysis measures the hydrophilicity on the modified surfaces. The measurements were carried out using a contact angle goniometer CAM 200 (KSV instruments Ltd, Helsinki, Finland). Measurements were performed at room temperature with water as a probe liquid. The droplet volume of 6.5 μl was used with a recording time of 120 sec for measuring the time dependency of the contact angle. The contact angles were measured on the three different spots on each sample.

The contact angles were seen to decrease rapidly as a function of oxidation time. The contact angle of the unmodified NFC-film was 44°, i.e. similar to the values reported elsewhere (see Spence K., et al., 2010). However, after only 10 seconds of oxidation, the contact angle was reduced from 44° to 19° indicating quite hydrophilic surface. Furthermore, prolonged oxidation (over 30 seconds) was found to produce extremely hydrophilic surfaces pushing contact angle values well below the detection limit of 15°. This demonstrates the fast reaction kinetics of TEMPO-oxidation which allows for rapid carboxylation of NFC-film. The contact angles of the carboxylated NFC-films seemed to be even lower than in films which were prepared from TEMPO-oxidized cellulose nanofibrils (pre-oxidation; see Fukuzumi H., et al., 2009). A plausible reason for this could be the high surface carboxyl content of the NFC-film, and peeling reactions that have been found to open the structure of cellulose microfibrils increasing the effective surface area (see Hirota M., et al., 2010).

The changes on the total charge due to the TEMPO-oxidation were followed by conductometric titration (FIG. 3). This procedure measures the increase in the charged groups (carboxyls) on the NFC films after the TEMPO treatment. It is important to note here that only the total extent of weak acid groups (only carboxyls are observed) can be measured by the conductometric method.

The analysis was carried out using a conductometric titrator 751 GPD Titrino (Metrohm AG, Herisau, Switzerland) following the standard SCAN-CM 65:02. The NFC-films (size 2.5×2.5 cm²) were acid washed with 0.01 M HCl for 1 hour, and disintegrated in MilliQ-water with a blade type homogenizer Polytron PT 2000 (Kinematica Inc., NY, USA). The conductometric titration was performed by adding 0.02 ml of 0.1 M NaOH within 30 sec intervals. The amount of weak acid groups (carboxyls) was calculated as is described in the standard (SCAN-CM 65:02).

The charge of the unmodified NFC-film was 72 μeq/g (reference), most likely due to the presence of hemicelluloses deriving from the hardwood pulp used for the NFC manufacturing process (see Pääkkö M., et al., 2007). This charge was elevated more than two-fold (169 μeq/g) even after a short 30 sec oxidation period. Moreover, the charge curve from FIG. 3 reveals that after 2 min of oxidation, the charge increase slows down to a plateau, indicating complete oxidation. The highest charge was found after 5 min oxidation (237 μeq/g) was interpreted to be close to the maximum carboxyl content that can be achieved without breaking the film's structure. This maximum was significantly lower than that of the individual TEMPO-oxidized nanofibrils (charge 1500 μeq/g; see Saito T., et al., 2006), strongly suggesting that the carboxylation reaction mainly occurred at the surface of the NFC-film. The aldehyde content of the NFC-film was not measured since only the carboxyl groups were utilized for the following EDC/NHS activation reaction (see Hermanson G. T., 2008).

The water resistance of TEMPO-oxidized NFC-films was tested by immersing them in water for several hours. The films remained intact thus suggesting that the carboxyl formation likely occurred only on the surface of a NFC-film because the oxidation inside the film would result in swelling and subsequent disintegration of the film.

The activation of the TEMPO-oxidized NFC was first verified by using the on-line QCM-D technique with NFC-model surfaces. The frequency shifts observed in a QCM are related to the gain or loss of mass on the sensor. However, it is important to consider that the changes in frequency are also affected by water coupling to the film. Note that the data range on the frequency axis in the QCM isotherms is the negative change in frequency; so in these profiles an increment of the signal indicates mass being increased (adsorption or water coupling) and a decrease of the signal indicates the loss of mass (desorption or hydrolysis). The NFC model surfaces were first carboxylated for 2 min by feeding a solution containing the chemicals needed for TEMPO-oxidation. This treatment caused an instant drop on the QCM frequency curve that is likely related to the formation of new carboxyl groups with increased amount of coupled water (FIG. 4a). It should be mentioned here that simultaneously recorded dissipation curve also raised drastically, which is indicative of thickened layer of coupled water on the NFC-surface (data not shown). As expected, the MilliQ-water rinsing further decreased the frequency values (change from −75 Hz to −130 Hz) and increased the dissipation values (change 35×10⁻⁶). This indicates that the removal of salts and other chemicals from the bulk phase is neutralizing the charge which in turn promotes the swelling of the NFC-film. The mass of the coupled water after TEMPO-oxidation and MilliQ-water rinsing was found to be 25.3 mg/m², which corresponds to the change of thickness of 25.3 nm if the water layer is assumed to be on a flat surface. However, the addition of sodium acetate buffer reduced the swelling of the NFC film which appears as an increase in the frequency curve. The EDC/NHS treatment of carboxylated NFC-surface decreased both the frequency (change over −25 Hz after buffer rinsing) and the dissipation values. This demonstrates the formation of NHS-esters and simultaneous decrease in anionic charge which reduces the swelling of the NFC-film. After buffer rinsing, the frequency and the dissipation curves quickly reached the plateau levels, which indicates stable attachment of NHS-esters on the NFC-surface. No desorption of NHS-esters was observed during the extended rinsing period. Next, the activated NFC-film was dried with nitrogen gas and stored overnight in a desiccator in prior to the reactivity test with BSA.

The TEMPO-oxidized NFC films before and after EDC/NHS activation were also analyzed by XPS. While the carboxylation of the NFC films could not be followed via XPS measurements (as reported by e.g. DiFlavio J., et al., 2007), most probably due to the medium-dependent surface adaptation of cellulose in dry medium (see Johansson L., et al., 2011), the esterification of the carboxyl groups leads to carbonyl groups, which are detectable (see e.g. Uschanov P., et al., 2011). Furthermore, the NHS-esters formed during film activation contain nitrogen not otherwise present in non-treated nor carboxylated film, so also nitrogen signal may be used as a fingerprint.

The topmost 10 nanometers of the films were examined using a Kratos Analytical AXIS 165 electron spectrometer with a monochromatic Al Kα X-Ray source at 100 W and a neutralizer. The XPS experiments were performed on the dry films, which were pre-evacuated overnight. At least three different spots of each sample were scanned. Spectra were collected at an electron take-off angle of 90° from sample areas less than one mm in diameter. Elemental surface compositions were determined from low-resolution measurements (80 eV pass energy and 1 eV step) while the surface chemistry was probed with high resolution measurements (20 eV pass energy and 0.1 eV step). The carbon C1s high-resolution spectra were curve fitted using parameters defined for cellulosic materials (see Johansson L. and Campbell J. M., 2004), and all binding energies were referenced to the aliphatic carbon component of the C1s signal at 285.0 eV (see Beamson G. and Briggs D., 1992). According to the in-situ reference (100% cellulose ash free filter paper), measured along with each sample batch, the conditions in UHV remained satisfactory during the XPS experiments (see Johansson L. and Campbell J. M., 2004).

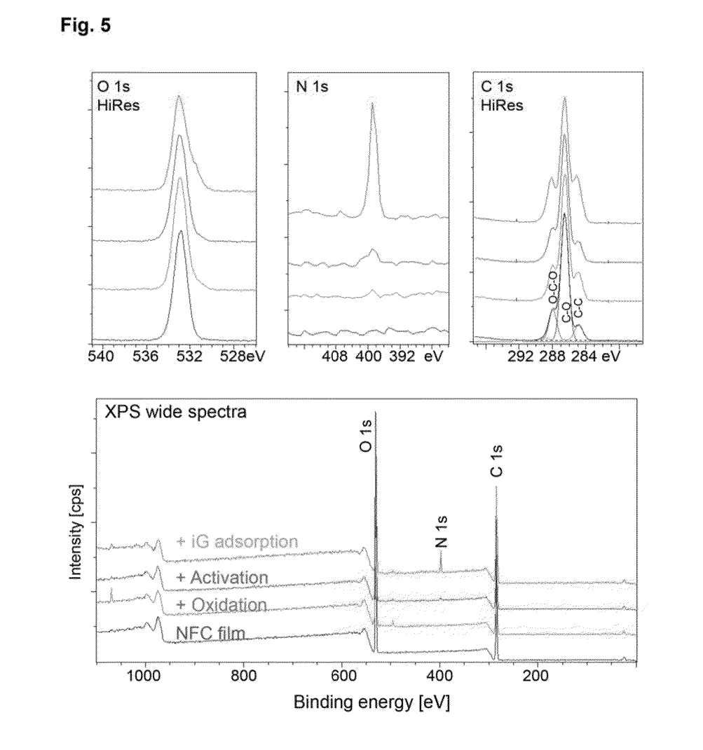

The XPS spectra in FIG. 5 shows that all the sample surfaces analysed were clean and had features characteristic of cellulose. Even the significant non-cellulosic C—C component in the non-modified and carboxylated films is in good accordance with other reports. However, after the EDC/NHS activation there is a clear nitrogen signal, accompanied by a similar increase in the C 1s carbonyl component (FIG. 5 and Table 1).

TABLE 1
XPS data for unmodified, TEMPO-oxidized, EDC/NHS activated TEMPO-oxidized
and EDC/NHS activated TEMPO-oxidized NFC-film with human IgG.

| | TEMPO | | | C 1s components (%) | | | | |
|---|---|---|---|---|---|---|---|---|
| | oxidation | O 1s | C 1s | C(C—C) | C(C—O) | C(C═O) | C(C O O) | N 1s |
| | time (sec.) | (at. %) | (at. %) | (%) | (%) | (%) | (%) | (at. %) |
| TEMPO-oxidized | 0 | 40.0 | 60.0 | 5.8 | 42.2 | 10.7 | 1.3 | 0.0 |
| NFC-films | 30 | 39.7 | 60.1 | 7.5 | 40.8 | 10.8 | 1.0 | 0.2 |
| | 60 | 38.5 | 60.9 | 8.9 | 39.2 | 11.8 | 1.1 | 0.1 |
| | 120 | 39.4 | 59.7 | 7.9 | 39.6 | 11.0 | 1.1 | 0.1 |
| | 300 | 38.6 | 60.3 | 9.5 | 38.9 | 10.8 | 1.1 | 0.2 |
| TEMPO-oxidized | 30 | 38.9 | 60.5 | 7.2 | 41.5 | 10.7 | 1.1 | 0.6 |
| plus EDC/NHS | 60 | 38.9 | 60.4 | 7.7 | 41.1 | 10.5 | 1.1 | 0.7 |
| activated NFC-films | 300 | 39.9 | 57.9 | 7.2 | 38.9 | 10.4 | 1.4 | 2.2 |
| Human IgG | 300 | 31.8 | 62.3 | 14.3 | 34.9 | 12.1 | 1.0 | 5.8 |
| adsorbed on | | | | | | | | |
| activated NFC-films | | | | | | | | |

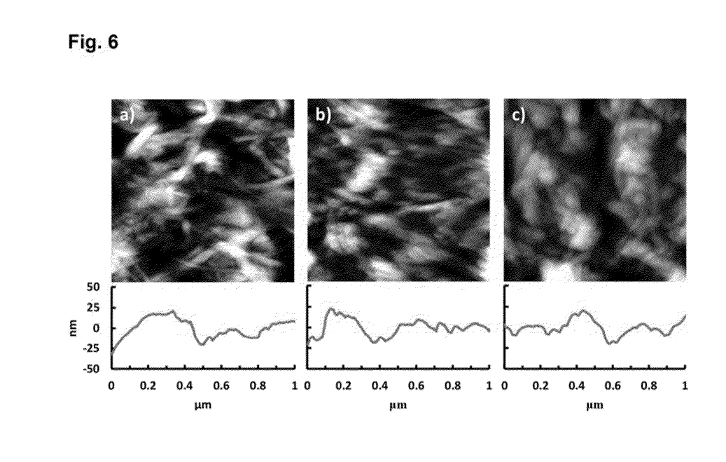

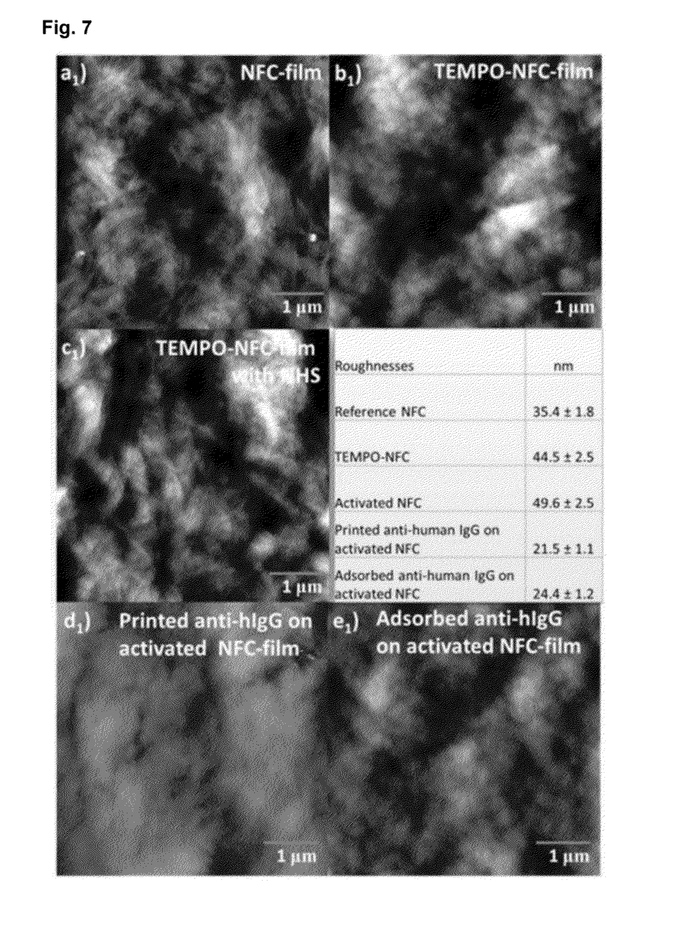

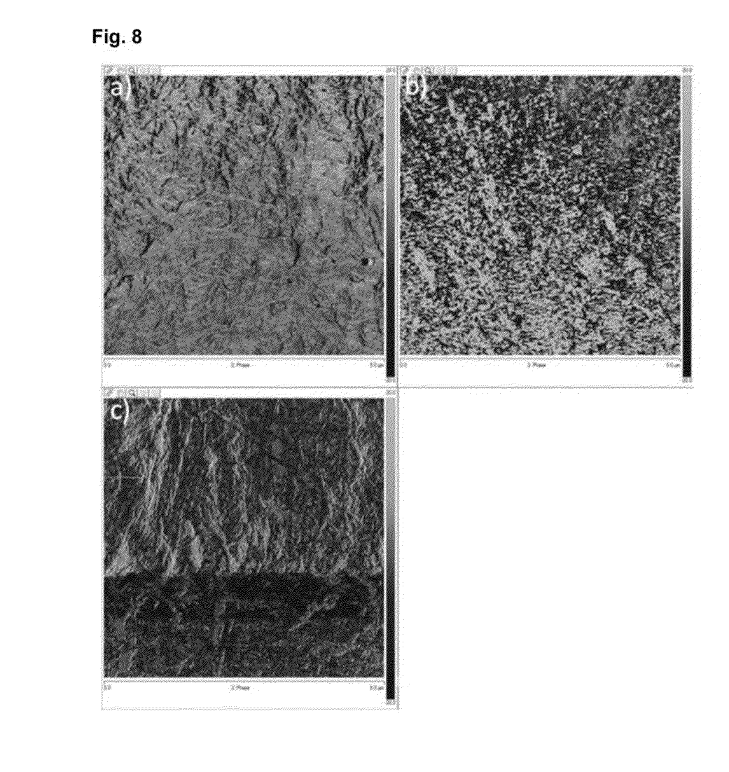

The topographical changes at a nanoscale (images of 1×1 μm²) were characterized by the AFM (FIG. 6a-c). In addition, images of 5×5 μm² are shown in the FIG. 7. On the unmodified NFC-film were cellulose nanofibril clusters clearly observable. Those clusters were originated from the mechanical homogenization that does not produce individual nanofibrils as can be obtained using the TEMPO-method (see Saito T. and Isogai A., 2004). No indications of large voids or pores were observed, which indicates that the natural affinity between cellulose fibrils lead to formation of a rather evenly distributed film. The roughness of the unmodified NFC-film was about 35.4 nm. After the TEMPO-oxidation the surface of the NFC-film was only slightly altered. The fibrillar structure was covered by a gel like layer that was also observable in the increased phase difference (see FIG. 8) corresponding to softening of the surface. Presumably this gel like structure was caused by carboxylated cellulose nanofibrils, which binds significant amount of water (see QCM-D data FIG. 4a) also from the room atmosphere, clinging the AFM tip to the film surface. The roughness of the TEMPO-oxidized NFC-film was about 44.5 nm. After NHS-activation, the fibrillar structure was clearly covered by coupled NHS-molecules. The EDC/NHS activation removes the charge of the NFC-film reducing the water binding capacity that can be seen in FIG. 7c, where the surface is similar compared to the unmodified NFC-film. The roughness of the activated NFC-film was about 49.6 nm. These results demonstrate that the coupled NHS-molecules were present on the NFC-film after the drying period.

The interaction and reaction analyses on NFC-surfaces were carried out in a QCM-D E4 instrument (Biolin Scientific AB, Gothenburg, Sweden). The principle of the QCM-D technique is described more detailed elsewhere (see e.g. Höök F., et al., 1998; Rodahl M., et al., 1995). All measurements were performed using a constant 100 μl/min flow rate, fixed 25° C. temperature, and all tests were repeated at least twice. The mathematical modeling to calculate the mass adsorption on the NFC-surfaces were carried out using the Johannsmann's model as is described by Johannsmann D., et al. (1992). Johannsmann's model is an iterative model using frequency overtones 3, 5, and 7 of the QCM-D crystal.

The TEMPO-mediated oxidation reaction with NFC-surfaces was tested in the QCM-D. The TEMPO-solution (0.13 mmol TEMPO, 4.7 mmol NaBr, and 5.65 mmol NaClO in MilliQ-purified water with fixed pH of 10) was allowed to flow over NFC-surfaces for 2 min. The oxidation reaction was then stopped by adding ethanol in the activation solution (10% from the volume of the activation solution), and then the solution was allowed to flow 2 min through the QCM-D chambers that interrupted the carboxylation reaction. Then the carboxylated NFC-surfaces were rinsed with a MilliQ-water treatment for 20 min to remove carboxylation chemicals. Finally, the surfaces were stabilized in 10 mM NaOAc at pH 5. The carboxylated NFC-surfaces were activated with a mixed solution of 0.1 M EDC with 0.4 M NHS in 10 mM NaOAc at pH 5 for 20 min that converts the carboxyl groups of TEMPO-oxidized NFC to amine reactive NHS-esters. Unused activation chemicals were then rinsed out with a buffer rinsing. The activated NFC-surfaces were finally dried with nitrogen gas, and stored in a desiccator if is not otherwise stated.

### Example 4

### Reactivity and Interaction of the Activated Films

To demonstrate the amine reactivity of activated NFC, bovine serum albumin (BSA) was chosen to provide a weight marker that is easily detectable in QCM-D monitoring. BSA (0.1 mg/ml) was adsorbed at pH 5 on the unmodified, TEMPO-oxidized and NHS-activated NFC-surfaces and the resulting surfaces were sequentially rinsed with NaOAc (pH 5), 10 mM NaCl (pH 10) and NaOAc (pH 5) buffer solutions, respectively (FIG. 4b). The control experiment with unmodified NFC-surface revealed irreversible binding of BSA as observed by negative frequency shift (−8 Hz, 1.6 mg/m²). This is not totally surprising since at the isoelectric point (pI) of BSA (ca. pH of 5) rather large amounts of this biomolecule have been found to adsorb on cellulose surfaces (see Orelma H., et al., 2011). In general, similar observations for the adsorption of BSA on non-cellulosic surfaces have been noted elsewhere as a result of reduced solvency at the pI (see Su T. J., et al., 1998; and Malmsten M., et al., 1998). However, it is noteworthy that alkaline buffer rinsing did not remove the bound BSA as could have been expected based on the electrostatic repulsion of negatively charged BSA and negatively charged NFC-surface. One possible explanation is the low negative charge of an unmodified NFC-surface.

Adsorption of BSA on a TEMPO-oxidized NFC-surface was found to be significantly higher (−35 Hz, 6.8 mg/m²) than that on the unmodified NFC-surface. However, the binding of BSA was reversible as the alkaline buffer rinsing completely removed the bound protein. This is likely due to the electrostatic repulsion between the negatively charged BSA (pI is 5; see Böhme U. and Scheler U., 2007) and negatively charged carboxylated TEMPO-oxidized NFC-surface (pI of carboxyls about 4.5; see Hoogendam C. W., et al., 1998). Furthermore, this demonstrates the regenerability of the carboxylated NFC-surface. The adsorption of BSA on TEMPO-oxidized NFC-surface was also tested at pH 7.4 and no adsorption was observed because of the electrostatic repulsion forces (data not shown). The initial adsorption of BSA on NHS-activated NFC-surface was found to be higher (−50 Hz, 9.8 mg/m²) than that on unmodified and TEMPO-oxidized NFC-surfaces. This can be explained by the esterification of free carboxylates which in turn reduces the negative charge of the NFC-surface, increasing the hydrophobicity of the surface that is beneficial for the adsorption of proteins (see Martin M. J., 1998). It can be seen from FIG. 4b that even after alkaline buffer rinsing approximately half (−25 Hz, 4.9 mg/m²) of the initially adsorbed BSA remains on the NFC-surface. This is most likely due to the hydrolyzation of NHS-esters groups in alkaline condition that increases the negative charge of the activated NFC-surface (see Hermanson G. T., 2008; Staros J. V., et al., 1986). This anionic charge with anionic charged proteins causes an electrostatic repulsion that removes electrostatically bound proteins. This clearly demonstrates that the EDC/NHS assisted activation of TEMPO-oxidized NFC-surface creates a topographical amine reactive platform, which can be used for the covalent conjugation of amine-bearing biomolecules.

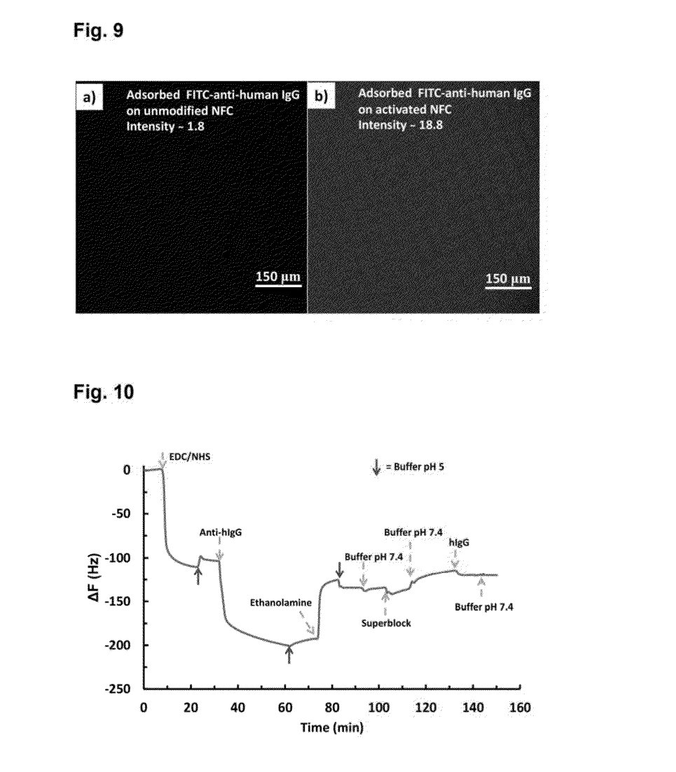

The attachment of antibodies on the activated NFC-films was investigated by using an adsorption method with FITC-staining by the CLSM (FIG. 9). The fluorescence imaging was carried out using laser wavelength 488 nm, where fluorescein produces an emission at wavelength around of 520 nm. In addition, no any auto-fluorescence of activated NFC was observed. The 0.1 mg/ml stained anti-human IgG was allowed to adsorb on the unmodified NFC- and the activated NFC-film. The surfaces were then treated with ethanolamine and 10 mM NaCl at pH 10, respectively, for remove unused NHS-groups and electrostatically bound antibodies. On the unmodified NFC-film the adsorption of anti-human IgG was low (FIG. 9a), which can be seen in the small fluorescence emission (intensity is about 1.8). Whereas, on the activated NFC-surface (FIG. 9b) the bounding was drastically higher (intensity is about 18.76). This result demonstrates that the activation of the NFC-surface led to stable conjugation of antibodies. Whereas, electrostatically bound anti-human IgG was washed out from the unmodified NFC-film in the pH 10 solution washing. In addition, the lattice type marking was observed on the fluorescence image (FIG. 9b), which is originated from the manufacturing process of the NFC-film. The immobilization of anti-human IgG on surface activated NFC-films was also verified by XPS (see the above FIG. 5), where nitrogen was used as a fingerprint. Unstained anti-human IgG was adsorbed on activated NFC-film similarly as is presented above. The film was extensively rinsed with ethanolamine and pH 10 solution for removing electrostatically bound antibodies. The clear increase in the nitrogen content (up to 6 at %, see the above Table 1) confirms the presence of antibodies on NFC-films (FIG. 5). These results again verified the influence of the topographical activation of the NFC-films to conjugate irreversibly antibodies on the NFC-film.

The reactivity of the activated NFC-surfaces after a drying period (stored in a desiccator for one day) was tested in QCM-D. The pre-activated NFC-surfaces were first stabilized in 10 mM NaoAc at pH 5 for 1 hour, and then 100 μg/ml BSA in 10 mM NaoAc at pH 5 was adsorbed on the activated NFC-surface for 30 min. The conjugation reaction was confirmed by a sequential rinsing sequence; pH 5 NaOAc buffer, 10 mM NaCl at pH 10, and pH 5 NaOAc buffer, respectively. The alkaline buffer rinsing causes an electrostatic repulsion between BSA and carboxylated NFC that removes the electrostatically bound protein. The adsorption of 100 μg/ml BSA in 10 mM NaOAc at pH 5 on the unmodified and TEMPO-oxidized NFC-surfaces was tested as a reference similarly as is described above.

The conjugation of anti-human IgG on activated NFC-surfaces was verified in QCM-D. The NFC surfaces were first activated similarly as is presented above, and then 100 μg/ml anti-human IgG in 10 mM NaAc at pH 5 was allowed to adsorb on the activated NFC-surface for 30 min followed the buffer rinsing. Unused NHS-esters were then removed by rinsing with 0.1 M ethanol amine at pH 8.5 for 15 min. The non-specific binding of proteins on a biointerface was blocked by a 15 min Superblock treatment. The activity of the conjugated antibody of a biointerface was tested by adsorbing 100 μg/ml hIgG in 10 mM phosphate buffer at pH 7.4 for 10 min on the biointerfaces with and without conjugated anti-human IgG. The non-specific adsorption of anti-human IgG and human IgG on unmodified and TEMPO-oxidized NFC surfaces were tested at similar conditions as is presented above.

QCM-D was exploited to monitor the conjugation reaction of anti-human IgG on the activated NFC-surface and subsequent adsorption of hIgG (FIG. 10). The NFC-surface was first TEMPO-oxidized for 2 min and then activated by adding the EDC/NHS reagent solution. In next step, a solution of anti-human IgG (0.1 mg/ml) was adsorbed on the NHS-activated NFC-surface and rinsed with given buffer solution. The initial adsorbed amount of anti-human IgG after buffer rinsing was found to be approximately −90 Hz (18 mg/m²). However, the addition of ethanolamine (pH 8.5) significantly decreased the adsorbed amount which was leveled off to about −23 Hz (4.6 mg/m²) after ethanol amine treatment representing the amount of the conjugated anti-body.

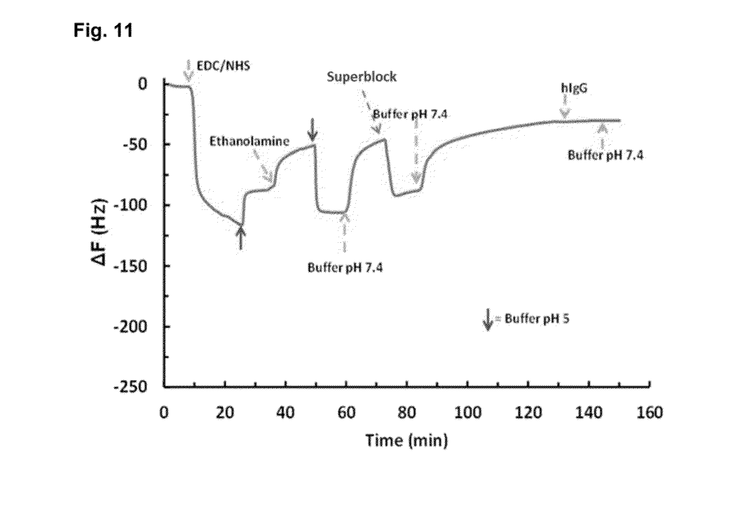

The desorption of antibody during ethanolamine treatment is most likely caused by the conversion of unused carboxyls to corresponding ethylamides, which are somewhat hydrophobic. The increase in hydrophobicity may reduce the specificity of the biointerface due to the hydrophobic interactions between the surface and the proteins, but this was prevented by adsorbing a blocking agent, superblock, before antigen detection to reduce the adsorption of cationically charged hIgG (pI about 8; see Hamilton R., 1987) on the anionic NFC-surface. The adsorption of superblock on the surface where anti-human IgG was installed was low, indicating that the anti-human IgG was covered remarkably the given surface. On the reference surface, without anti-human IgG (see FIG. 11), the frequency change was much larger demonstrating that the superblock treatment altered also the amount of coupled water. As a reference, and to demonstrate the amount of non-specific binding, human IgG was adsorbed to ethanolamine treated NHS-activated NFC-surface. As can be seen from FIG. 11 the final amount of adsorbed human IgG to a reference surface is significantly lower than that of the anti-human IgG containing surface. This clearly demonstrates the effectiveness of covalently conjugated anti-human IgG in capturing the human IgG from a solution. The 0.1 mg/ml human IgG was adsorbed on the anti-human IgG interface and on the reference surface (without anti-human IgG) at pH 7.4. Human IgG adsorbed on the surface, where anti-human IgG was installed (adsorption about −5 Hz, 0.93 mg/m²), whereas on the reference surface no any adsorption was observed. These experiments demonstrate that after the conjugation of anti-human IgG on the NFC-surface via TEMPO-oxidation the antibody was detected specifically. Moreover, the negligible non-specific adsorption of human IgG on the NFC-surface after the superblock treatment enabled the use of NFC-films in immunoassay applications where positively charged target molecules are monitored. The amount of adsorption of human IgG on the anti-human IgG interface was over two-fold higher compared to earlier published data where anti-hIL-1ra antibodies were covalently conjugated on CMC-PEI hydrogel in QCM-D (see Carrigan S. D., et al., 2005).

### Example 5

### Inkjet Printing and Adsorption on Activated NFC-Films

The inkjet printing of antibodies on activated NFC-films was demonstrated by using an EPSON R800 inkjet printer. 1 mg/ml fluorescence (dansyl or FITC-probe) stained anti-human IgG in 10 mM phosphate buffer at pH 7.4 was printed on the activated NFC-films (size 2×4 cm²) using a CD printing tray with an EPSON print-CD software. The printing conditions were selected using a CMYK color profile that allows printing from a selected cartridge. All other cartridges were filled with 10 mM phosphate buffer at pH 7.4. The anti-human IgG was fluorescence stained using fluorescence dyes of dansyl and FITC following procedures as is presented elsewhere (see Ronald B. J., 2001; Hermanson G. T., 2008). The stained antibody solution was further purified with Amicon Ultra centrifugal filter tubes (The cut of Mw of 30 kM) with three pass of phosphate buffer (total rinsing volume of 6 times of the original sample volume). Finally, the stained anti-human IgG was diluted to the final concentration with 10 mM phosphate buffer at pH 7.4, and it was printed instantly on activated NFC-films. The printed NFC-films were then treated with 0.1 M ethanol amine at pH 8.5 for 15 min, and dried at the room temperature. Printed fluorescence stained anti-human IgG on the activated NFC-films were illustrated under the UV-light (366 nm, Camag, Berlin, Germany) and with a CLSM instrument.

The attachment of anti-human IgG on the activated NFC-films was also examined by using a simple adsorption technique. 0.1 mg/ml FITC stained or unstained anti-human IgG in 10 mM phosphate buffer at pH 7.4 was allowed to adsorb on unmodified and activated NFC for 20 min. The films were then treated with 0.1 M ethanol amine at pH 8.5 for 15 min, and rinsed with 10 mM phosphate buffer at pH 7.4, and 10 mM NaCl at pH 10, respectively, to remove electrostatically bound antibodies. Finally the surfaces were rinsed with Milli-Q water and dried in the room temperature. The adsorbed FITC stained anti-human IgG on unmodified and activated NFC-films were imaged with a CLSM.

The conjugation of adsorbed FITC stained anti-human IgG on the activated NFC-films was verified with excitation wavelength of 488 nm and a detected wavelength range of 490-550 nm (no auto-fluorescence of unmodified NFC was observed in such measurement conditions). The intensity images were scanned using scanning mode with the averaging mode and constant imaging conditions (laser power was 848V in all measurements). The intensity of the florescence images were measured using an analysis tool of Photoshop (Adobe). The intensity was measured from the unmodified raw images.

The nanoscale topographical changes on the NFC-films were analyzed using a Nanoscope IIIa Multimode scanning probe microscope (Digital Instruments, Inc., Santa Barbara, Calif.). The images were scanned using the silicon cantilevers (Ultrasharp μmasch, Tallinn, Estonia). At least three different spots of each sample area were scanned with imaged sizes of 5×5 μm² and 1×1 μm². No any image processing was done excluding image flattening. The roughness profiles were calculated from the 5×5 μm² images.

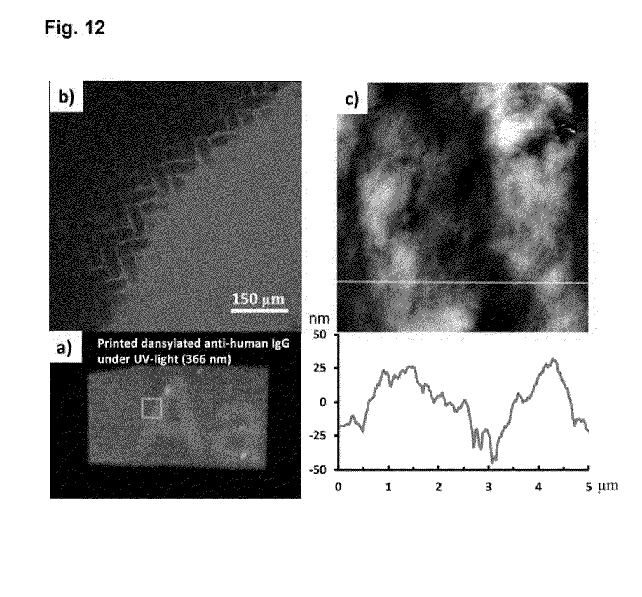

The attachment of anti-human IgG on the activated NFC-films in the larger scale was demonstrated using inkjet-printing and adsorption techniques. The 1 mg/ml dansyl stained anti-human IgG at pH 7.4 was printed on the activated NFC-film (FIG. 12a), and the film was then rinsed with ethanolamine to remove unused NHS-groups, which also removes partly the electrostatically bound antibodies. After drying, the dansyl stained anti-human IgG on the NFC-film was illustrated under the UV-light (366 nm), where dansyl has a blue fluorescence emission. It can be seen that the NFC-film produces significant auto-fluorescence under the UV-light (366 nm). However, the texture of the printed anti-human IgG was clearly observable indicating that printed antibodies were irreversible attached on the activated NFC-film. The inkjet printing of anti-human IgG on the activated NFC-film was further analyzed with FITC-staining by CLSM. After printing of the anti-human IgG texture on an activated NFC-surface, the surface was rinsed with ethanolamine and 10 mM NaCl at pH 10 for remove noncovalently bound antibodies. Due to higher magnification of a CLSM instrument only a border of printed texture can be shown. A clear border in the fluorescence intensity image was observed that demonstrates the covalent attachment of antibody on the NFC-film (FIG. 12b). The topographical changes on the activated NFC-film after printing of a texture of anti-human IgG were imaged by AFM (FIG. 12c). The AFM image shows that the surface was fully covered by antibody and no any cellulose microfibrilar structure was visible that verified the uniform printing layer on the NFC-film. The RMS roughness of the image was approximately 25 nm that was lower compared that of unmodified NFC-film (35 nm).

### REFERENCES

Wu, P.; Castner, D. G.; Grainger, D. W. Journal of biomaterials science. Polymer edition 2008, 6, 725-753.
Yager, P.; Edwards, T.; Fu, E.; Helton, K.; Nelson, K.; Tam, M. R.; Weigl, B. H. Nature 2006, 7101, 412-418.
Lequin, R. M. Clinical Chemistry 2005, 12, 2415-2418.
Siró, I.; Plackett, D. Cellulose 2010, 3, 459-494.
Pelton, R. TrAC Trends in Analytical Chemistry 2009, 8, 925-942.
Mansfield, M. A. The Use of Nitrocellulose Membranes in Lateral-Flow Assays. In Drugs of Abuse; Wong, R. C., Tse, H. Y., Eds.; Humana Press: 2005; pp 71-85.
Saito, T.; Kimura, S.; Nishiyama, Y.; Isogai, A. Biomacromolecules 2007, 8, 2485-2491.
Pääkkö, M.; Ankerfors, M.; Kosonen, H.; Nykanen, A.; Ahola, S.; Osterberg, M.; Ruokolainen, J.; Laine, J.; Larsson, P. T.; Ikkala, O.; Lindström, T. Biomacromolecules 2007, 6, 1934-1941.
Syverud, K.; Stenius, P. Cellulose 2009, 1, 75-85.
Yano, H.; Nakahara, S. J. Mater. Sci. 2004, 5, 1635-1638.
Karabulut, E.; Wagberg, L. Soft Matter 2011, 7, 3467-3474.
Spence, K.; Venditti, R.; Rojas, O.; Habibi, Y.; Pawlak, J. Cellulose 2010, 4, 835-848.
Nogi, M.; Iwamoto, S.; Nakagaito, A. N.; Yano, H. Adv Mater 2009, 16, 1595-1598.
Österberg, M.; Ly, T.; Vartiainen, J.; Lucenius, J.; Hippi, U.; Seppälä, J.; Serimaa, R.; Laine, J. ACS Appl. Mater. Interfaces, 2013, 5 (11), 4640-4647
Norde, W. Macromolecular Symposia 1996, 1, 5-18.
De Nooy, A.; Besemer, A. C.; Van Bekkum, H. Carbohydr. Res. 1995, 1, 89-98.
Isogai, A.; Kato, Y. Cellulose 1998, 5, 153-164.
Saito, T.; Nishiyama, Y.; Putaux, J.; Vignon, M.; Isogai, A. Biomacromolecules 2006, 6, 1687-1691.
Saito, T.; Isogai, A. Biomacromolecules 2004, 5, 1983-1989.
Habibi, Y.; Chanzy, H.; Vignon, M. Cellulose 2006, 6, 679-687.
Fukuzumi, H.; Saito, T.; Iwata, T.; Kumamoto, Y.; Isogai, A. Biomacromolecules 2009, 1, 162-165.
Abe, K.; Suzuki, K.; Citterio, D. Analytical Chemistry 2008, 18, 6928-6934.
Hossain, S. M.; Luckham, R. E.; Smith, A. M.; Lebert, J. M.; Davies, L. M.; Pelton, R. H.; Filipe, C. D.; Brennan, J. D. Analytical Chemistry 2009, 13, 5474-5483.
Di Risio, S.; Yan, N. J. Adhes. Sci. Technol. March 2010, 661-684.
Lonini, L.; Accoto, D.; Petroni, S.; Guglielmelli, E. J. Biochem. Biophys. Methods 2008, 6, 1180-1184.
Delaney, J. T.; Smith, P. J.; Schubert, U. S. Soft Matter 2009, 24, 4866-4877.
Rose, D. Drug Discov. Today 1999, 9, 411-419.
Ahola, S.; Myllytie, P.; Österberg, M.; Teerinen, T.; Laine, J. Bioresources 2008, 4, 1315-1328.
Johansson, L.; Campbell, J. M. Surf. Interface Anal. 2004, 8, 1018-1022.
Beamson, G.; Briggs, D. High resolution XPS of organic polymers; Wiley: Chichester, UK, 1992; pp 295.
Höök, F.; Rodahl, M.; Brzezinski, P.; Kasemo, B. Langmuir 1998, 4, 729-734.
Rodahl, M.; Hook, F.; Krozer, A.; Brzezinski, P.; Kasemo, B. Rev. Sci. Instrum. 1995, 7, 3924-3931.
Johannsmann, D.; Mathauer, K.; Wegner, G.; Knoll, W. Phys. Rev. B 1992, 12, 7808-7815.
Ronald, B. J. Pharmacol. Toxicol. Methods 2001, 3, 247-253.
Hermanson, G. T. Bioconjugate techniques; Academic Press: San Diego (Calif.), 2008; Vol. 2, pp 1202.
Hirota, M.; Furihata, K.; Saito, T.; Kawada, T.; Isogai, A. Angewandte Chemie 2010, 42, 7836-7838.
DiFlavio, J.; Pelton, R.; Leduc, M.; Champ, S.; Essig, M.; Frechen, T. Cellulose 2007, 3, 257-268.
Johansson, L.; Tammelin, T.; Campbell, J. M.; Setala, H.; Osterberg, M. Soft Matter 2011, 22, 10917-10924.
Uschanov, P.; Johansson, L.; Maunu, S.; Laine, J. Cellulose 2011, 2, 393-404.
Orelma, H.; Filpponen, I.; Johansson, L.; Laine, J.; Rojas, O. J. Biomacromolecules 2011, 12, 4311-4318.
Su, T. J.; Lu; Thomas, R. K.; Cui, Z. F.; Penfold, J. The Journal of Physical Chemistry B 1998, 41, 8100-8108.
Malmsten, M.; Emoto, K.; Van Alstine, J. M. Journal of Colloid and Interface Science 1998, 2, 507-517.
Böhme, U.; Scheler, U. Chemical Physics Letters 2007, 4-6, 342-345.
Hoogendam, C. W.; de Keizer, A.; Cohen Stuart, M. A.; Bijsterbosch, B. H.; Smit, J. A. M.; van Dijk, J. A. P. P.; van der Horst, P. M.; Batelaan, J. G. Macromolecules 1998, 18, 6297-6309.
Martin, M. J. Colloid Interface Sci. 1998, 2, 186-199.
Staros, J. V.; Wright, R. W.; Swingle, D. M. Analytical Biochemistry 1986, 1, 220-222.
Hamilton, R. Clin Chem 1987, 33, 1070-1075.
Carrigan, S. D.; Scott, G.; Tabrizian, M. Langmuir 2005, 13, 5966-5973.

1. A method for the topographical modification of the surface of a nanofibrillar cellulose (NFC) film, characterized by using TEMPO oxidation and carboxylation of the film surface to provide a surface-reactive NFC film.

2. The method according to claim 1, wherein the NFC film used as the substrate is prepared from mechanically disintegrated cellulose gel.

3. The method according to claim 1, wherein one or more further surface modification steps are carried out subsequent to the carboxylation.

4. The method according to claim 3, wherein the carboxyl groups are further reacted into ester groups using an EDC/NHS reaction as a further surface modification step.

5. The method according to claim 3, wherein amine-containing molecules are applied onto the surface of the NFC film as a further surface modification step, the amine-containing molecules preferably being selected from biomolecules, more preferably from proteins, DNA and antibodies, even more preferably from either water-soluble proteins, such as albumins, or from monomeric immunoglobulins, such as IgG, IgD and IgE, most suitably from bovine serum albumin (BSA) or an immunoglobulin IgG.

6. The method according to claim 3, wherein amine-containing molecules are attached to the carboxyl groups on the surface of the NFC film as a further surface modification step.

7. The method according to claim 3, wherein amine-containing molecules are applied onto the surface of the NFC film using non-contact application methods, preferably selected from inkjet printing, spraying and adsorption techniques.

8. The method according to claim 1, which is carried out entirely in aqueous media.

9. A nanofibrillar cellulose (NFC) film with a topographically modified surface, characterized in that it has been prepared using the method according to claim 1.

10. Use of the method according to claim 1 in preparing barrier films, bioactive indicators, or affinity filters, particularly in preparing a platform for diagnostical assays or a biofilter.

11. Use of the method according to claim 1 in functionalizing cellulosic materials other than NFC, such as paper, films, or membranes.
